(19) United States
(12) Patent Application Publication
(10) Pub. No.: US 2020/0275986 A9
(43) Pub. Date: Sep. 3, 2020

(54) SUCTIONING DEVICE

(71) Applicant: Sanverbos, Inc., Sonoma, CA (US)

(72) Inventor: Sean NORRIS, Sonoma, CA (US)

(21) Appl. No.: 15/952,564

(22) Filed: Apr. 13, 2018

Related U.S. Application Data
(60) Provisional application No. 62/485,826, filed on Apr. 14, 2017.

(51) Int. Cl. A61B 42/10; B29D 99/00; A61M 1/00; A61B 5/00

(52) U.S. Cl. CPC A61B 42/10; B29D 99/0067; A61M 2209/088; A61B 5/6806; A61B 2217/005; A61M 1/008

(57) ABSTRACT

A medical glove is disclosed that includes a suctioning device that is integrated into the glove. The medical glove may have one or more orifices at an end and may include a channel. In one example, the medical glove may be formed using a hand former with pre-formed channel. The channel may include one or more biosensors.

This patent application claims priority to Provisional Application No. 62/485,826 filed Apr. 14, 2017, the disclosure of which is incorporated herein in their entirety.

### BACKGROUND OF THE INVENTION

### 1. Field of the Invention

The present disclosure is directed to a medical glove with an incorporated suction device. A medical glove with a suction device incorporated within the material of the medical glove along the index or multiple fingers will allow the operating surgeon or dentist the use of both hands and the ability to suction during a procedure while retaining flexibility for dexterity and precise manipulation at his fingers.

### 2. Background

Medical gloves, which include both surgical and patient examination gloves, are used to prevent transmission of a wide variety of diseases, such as bacterial or viral infections, to both patients and health care personnel such as surgeons, dentists, veterinarians, and health care workers. While patient examination gloves are intended to prevent contamination between the patient and the examiner, surgical gloves are worn by operating room personnel to protect a surgical wound from contamination.

Additionally, during dental or medical procedures, suctioning devices are used to remove fluids, such as saliva, or blood, from the area being operated on to allow surgeons to view and work on the area, also free of debris such as tissue, bone, or amalgam. For example, in some dentistry procedures such as cavity fillings, it is important that the tooth stay clean and dry, so the suction removes any saliva, blood, and water from dental drills that accumulate around the tooth.

Often when suction is used during dental or medical procedures, the operating surgeon or dentist must hold the suction himself or an assistant must be present to perform the suctioning. However, it is desirable for the operating surgeon's or dentist's hands to be free to perform other important tasks while still maintaining the ability to suction. Additionally, current suction instruments are imprecise extensions of the operating surgeon's or dentist's hand in comparison to the operating surgeon's or dentist's own fingers which are controlled with precision, dexterity, and flexibility.

### SUMMARY

In one aspect, there is disclosed a medical glove comprising a cuff having an opening for hand entry and an opening edge, a body section coupled to the cuff; a plurality of finger members coupled to the body section generally opposite from the cuff, a first channel integrally formed in the glove, where the first channel includes a first open end proximate to the opening edge, a second open end proximate to the tip end of one of the finger members, and an enclosed hollow space connecting the first open end and the second open end, where the second open end comprises a plurality of orifices and at least one biosensor included in the channel.

The second open end may extend at least partially around the tip end of the finger member. The glove may further comprise a first material that forms the channel, and each of the plurality of orifices has generally the same shape and at least one of the plurality of orifices has a different shape or size than the other orifices.

In some embodiments, at least a portion of the orifices are arranged in two or more rows. The glove may form a second channel. In some embodiments, the second channel is positioned on one of the same finger member as the first channel and a different finger member than the first channel. The first channel may provide suction and the second channel may provide irrigation. The medical glove may include one or more supporting structures to prevent collapse of the first channel when the first channel is under suction. In some embodiments, the first channel further comprises a tube embedded into the glove.

In another aspect, a method of forming a medical glove includes forming a channel, placing the channel into a groove of a hand former, where the groove is configured for receiving the channel, beading a cuff of the hand former, drying or curing the hand former, removing the formed glove from the hand former, and laser cutting or machine punching one of end and side holes in the glove. The method may include one of leaching and vulcanizing the hand former.

The method may include forming the glove from one of latex and a synthetic polymer. The method may include forming the channel from one of dipping, extrusion, cast mold on silicone, injection molding, ceramic and a steel rod. The method may include a biosensor in the channel. The method may include one of analyzing and detecting biomarkers with the biosensor.

In another aspect, a hand former for a medical glove is disclosed including a body substantially in the shape of a hand and an opening at one of end of the body, and a groove extending from the opening substantially to or near the end of one or more fingers of the hand former and configured to receive a channel. The medical glove is formed from one of latex and a synthetic polymer. The hand former may also include a channel that is formed from one of dipping, extrusion, cast mold on silicone, injection molding, ceramic and a steel rod. In some embodiments, a biosensor is included in the channel.

### BRIEF DESCRIPTION OF THE DRAWINGS

The advantages of the invention and preferred embodiments are more fully understood by referencing the following detailed descriptions of the drawings.

FIG. 1 illustrates a side view of a medical glove with an integrated channel according to various embodiments.

FIG. 2 illustrates a front view of a medical glove with an integrated channel according to various embodiments.

FIG. 3 illustrates a side view of finger members of a medical glove with an integrated channel according to various embodiments.

FIG. 4 illustrates a side view of finger members of a medical glove with an integrated channel according to various embodiments.

FIG. 5 illustrates a side view of finger members of a medical glove with an integrated channel according to various embodiments.

FIG. 6 illustrates a side view of finger members of a medical glove with an integrated channel according to various embodiments.

FIG. 7 illustrates a support member according to various embodiments.

FIG. 8 illustrates a support member positioned at a channel on a finger member of a medical glove according to various embodiments.

FIG. 9 illustrates a cross-sectional view of a medical glove with an integrated channel according to various embodiments.

FIG. 10 illustrates a cross-sectional view of a medical glove with an integrated channel according to various embodiments.

FIG. 11 illustrates a cross-sectional view of a medical glove with an integrated channel according to various embodiments.

FIG. 12 illustrates a cross-sectional view of a medical glove with an integrated channel according to various embodiments.

FIG. 13 illustrates a side view of finger members of a medical glove with an integrated channel according to various embodiments.

FIG. 14 illustrates a perspective view of a suction head according to various embodiments.

FIG. 15 illustrates a perspective view of a suction head according to various embodiments.

FIG. 16 illustrates a side view of finger members of a medical glove with a first integrated channel and a second integrated channel according to various embodiments.

FIG. 17 is a flow diagram depicting a method for fabricating a medical glove with an integrated channel.

FIG. 18 is a flow diagram depicting an alternate method for fabricating a medical glove with an integrated channel

FIG. 19 is a flow diagram showing a method for fabricating a hand former in an embodiment.

FIGS. 20A-20B show a hand former in an embodiment.

FIG. 21 shows a process for forming a medical glove in an embodiment

### DETAILED DESCRIPTION

Each of the additional features and teachings disclosed below can be utilized separately or in conjunction with other features and teachings to provide a device, system, and/or method for medical glove including an integrated suction device. Representative examples of the present invention, which examples utilize many of these additional features and teachings both separately and in combination, will now be described in further detail with reference to the attached drawings. This detailed description is merely intended to teach a person of skill in the art further details for practicing preferred aspects of the present teachings and is not intended to limit the scope of the invention. Therefore, combinations of features and steps disclosed in the following detail description may not be necessary to practice the invention in the broadest sense and are instead taught merely to particularly describe representative examples of the present teachings.

Moreover, the various features of the representative examples and the dependent claims may be combined in ways that are not specifically and explicitly enumerated to provide additional useful embodiments of the present teachings. In addition, it is expressly noted that all features disclosed in the description and/or the claims are intended to be disclosed separately and independently from each other for original disclosure, as well as for restricting the claimed subject matter independent of the compositions of the features in the embodiments and/or the claims. It is also expressly noted that all value ranges or indications of groups of entities disclose every possible intermediate value or intermediate entity for original disclosure, as well as for restricting the claimed subject matter.

While this technology is susceptible of embodiment in many different forms, there is shown in the drawings and will herein be described in detail several specific embodiments with the understanding that the present disclosure is to be considered as an exemplification of the principles of the technology and is not intended to limit the technology to the embodiments illustrated.

The terminology used herein is for describing embodiments only and is not intended to be limiting of the technology. As used herein, the singular forms “a”, “an” and “the” are intended to include the plural forms as well, unless the context clearly indicates otherwise. It will be further understood that the terms “comprise” and/ or “comprising,” when used in this specification, specify the presence of stated features, integers, steps, operations, elements, and/or components, but do not preclude the presence or addition of one or more other features, integers, steps, operations, elements, components, and/or groups thereof.

It will be understood that like or analogous elements and/or components, referred to herein, may be identified throughout the drawings with like reference characters. It will be further understood that several of the figures are merely schematic representations of the present technology. As such, some of the components may have been distorted from their actual scale for pictorial clarity.

Embodiments of the present invention are directed to a medical glove with an incorporated suction device.

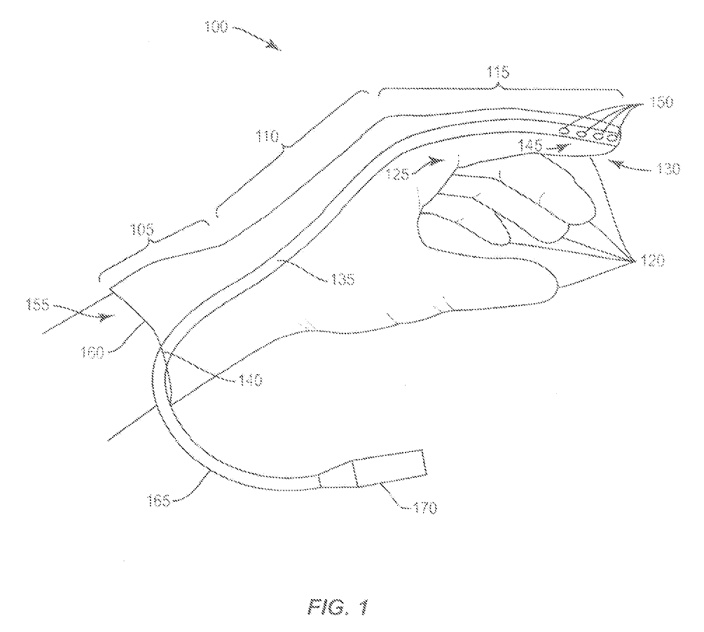

FIG. 1 illustrates an exemplary embodiment of a medical glove 100, comprising a cuff 105, a body section 110 coupled to the cuff 105, and a finger section 115. The cuff 105 may further comprise an opening 155 for hand entry, the opening 155 having an opening edge 160. The finger section 115 may comprise a plurality of individual finger members 120. Each finger member 120 comprises a base end 125 where the finger member 120 couples to the body section 110, and a tip end 130 opposite the base end 125. The body section 110 of the medical glove 100 may couple the cuff 105 and the finger section 115 and may generally cover a top of a hand and a palm of a user of the medical glove 100. The medical glove 100 may further comprise an enclosed hollow channel 135 beginning at a first open end 140 proximate to the cuff opening edge 160, extending continuously across the cuff 140, body section 110 and at least one finger member 120. The channel 135 may terminate at a second open end 145 proximate to the tip end 130 of the finger member 120. In various embodiments, the medical glove 100 may further comprise a suction tubing 165 within the channel 135 (see FIG. 12). The tubing 165 may extend beyond the cuff opening edge 160 as illustrated in FIG. 1 and terminate at a connector 170 to facilitate connection to a suction source. In various embodiments, the medical glove 100 may be made from synthetic or non-synthetic polymers such as latex, vinyl, nitrile, polyurethane, and the like.

Furthermore, the medical gloves 100 may be disposable, reusable, sterile, or not sterile for non-surgical uses. In various embodiments in which suction tubing 165 is incorporated within the material of medical glove 100, there is no separation between suction tubing 165 and the surrounding glove material. If a perforation is encountered in the medical glove 100 around suction tubing 165, an indicator glove underneath (not shown) may show any possible leakage.

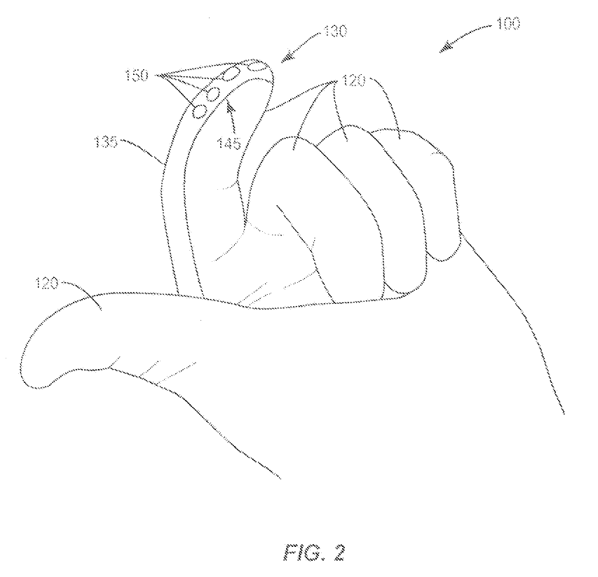

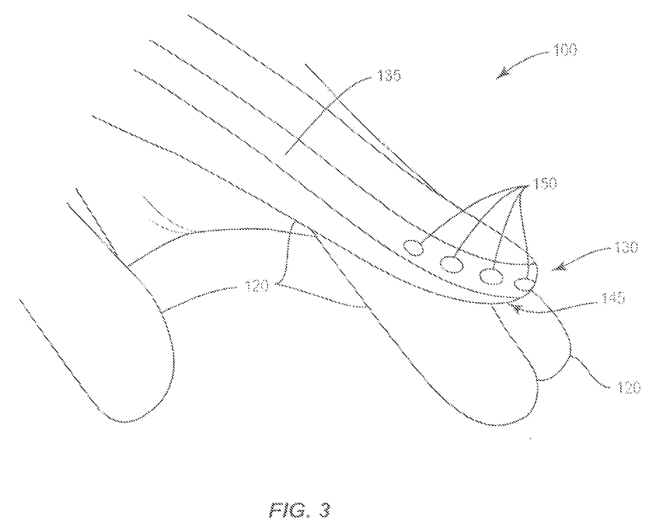

FIGS. 2 and 3 illustrates the second open end 145 of the channel 135 at the tip end 130 of the finger member 120, according to various embodiments in which the second open end 145 comprises a plurality of individual orifices 150. Although certain embodiments may comprise a single orifice 150, having a plurality of orifices 150 may better facilitate the second open end 145 to function as a pool suction device, thereby allowing suctioning of large volumes of fluid. Further, distributing the plurality of orifices 150 along a side of the fingertip 130 and at least partially around the fingertip 130 allows the user to easily position one or more of the orifices 150 precisely and accurately where needed with less overall movement of the user's finger. The plurality of orifices 150 may allow for continuous suction without clogging. In various embodiments, the orifices 150 may be any shape, size, or in any position at the tip end 130 of the finger member 120 that allows for continuous suction without clogging. For example, a dentist utilizing the medical glove 100 could maneuver his finger to suction an area accumulating saliva or dry the area around a tooth during an examination or procedure. Additionally, a general surgeon, for example, utilizing the medical glove 100 could maneuver his finger into a difficult to reach or non-visible area to suction blood or fluid from the obstructed surgical field.

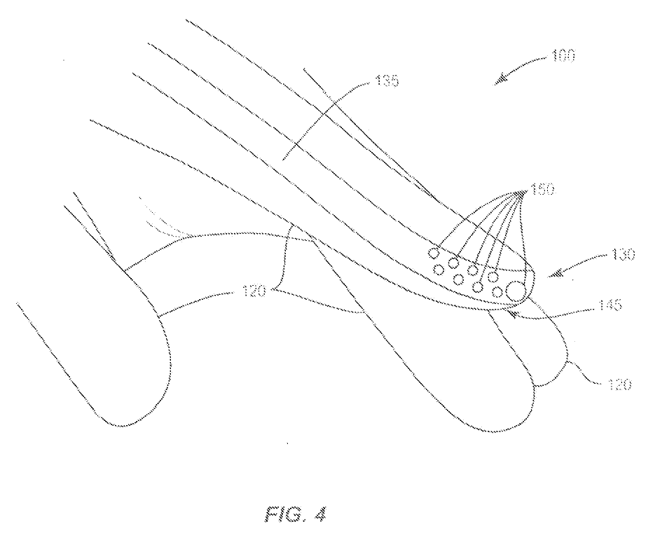

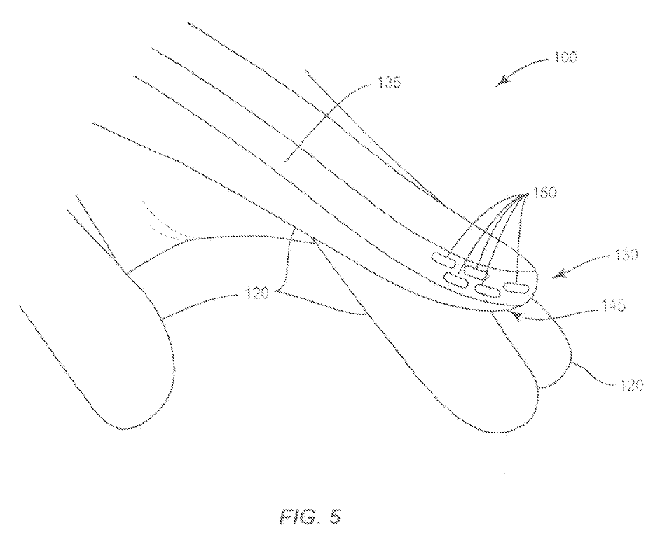

As mentioned above, the plurality of orifices 150 may be any shape or size and may be in any position in proximity to the tip end 130 of the finger member 120. FIGS. 4 through 6 illustrate certain nonlimiting examples of shape, size and positioning of a variety of orifices 150. Although FIGS. 1 through 3 illustrated a single row of generally equally sized orifices 150, FIG. 4 illustrates that various embodiments may comprise multiple rows and sizes of orifices 150 in combination. In the example of FIG. 4, a single larger orifice 150 may be positioned near the tip end 130. The larger orifice 150 may be useful in, for example, dental applications where solid material may need to be suctioned away but which may clog smaller orifices 150. The smaller orifices 150 in FIG. 4 may be used to suction away liquids. In FIG. 5, the orifices 150 comprise slots instead of generally round holes to further facilitate removal of solids or to facilitate the removal of larger volumes of liquid in a shorter period. FIGS. 4 and 5 also illustrated that multiple rows of orifices 150 may be combined with a single row of orifices 150. In further exemplary embodiments as illustrated in FIG. 6, a variety of shapes, sizes, and rows of orifices 150 may be used in combination. Thus, as will be clear to one skilled in the art from the various embodiments illustrated in FIGS. 1-6, any shape, size, orientation, or combination of orifices 150 is within the scope of the present disclosure.

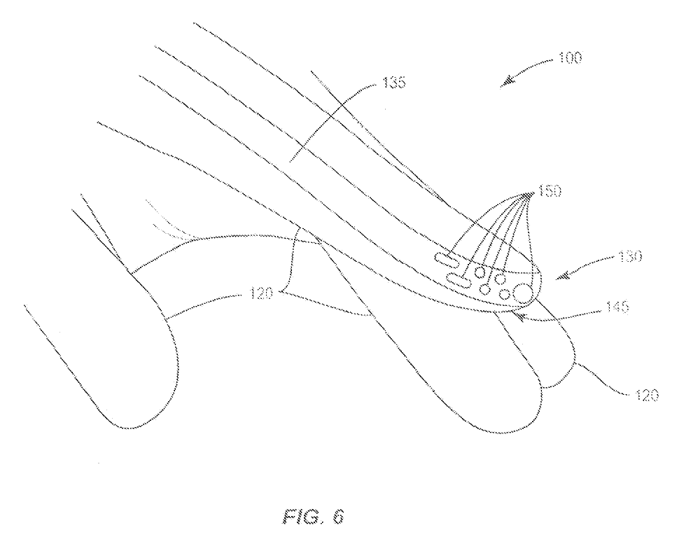

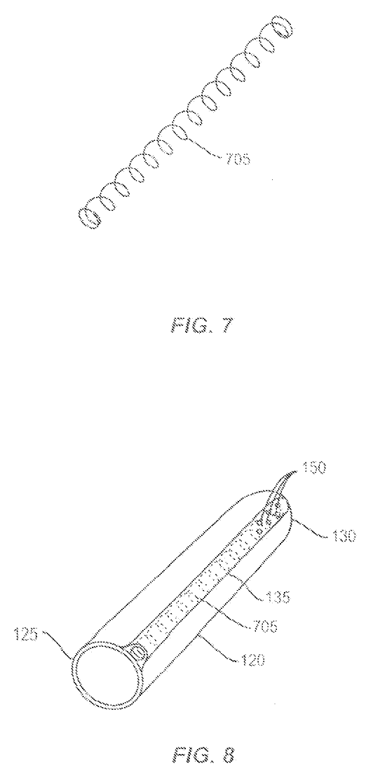

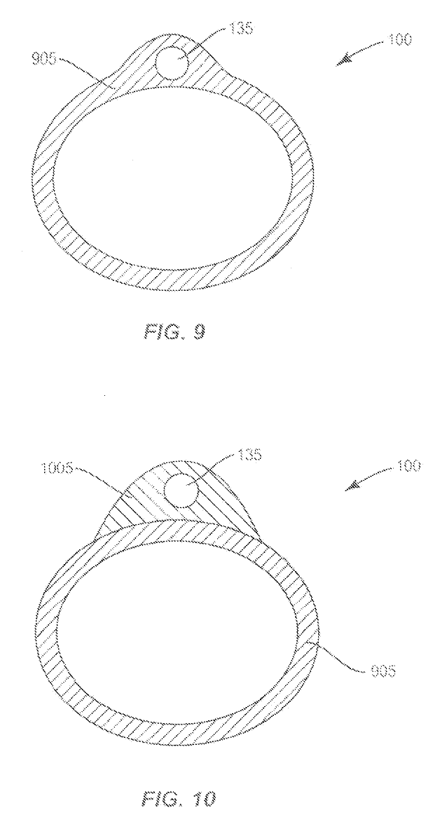

Placing the channel 135 under suction may tend to cause the channel 135 to at least partially collapse, thereby limiting the suctioning capability of the medical glove 100. FIG. 7 illustrates a reinforcing coil 705 that may be molded into sidewalls of the channel 135 or may be inserted into the channel 135. The coil 705 may comprise a flexible yet resilient material such as an elastomeric or other polymeric material to retain flexibility for dexterity and precise manipulation of the user's fingers. In certain embodiments, the coil 705 may comprise a metal, such as stainless steel or titanium. Additionally, the coil 705 may be of any length desired, and may run the entire length of the channel 135 or any portion or portions of the channel 135. In various embodiments, the coil 705 may be absent where finger joints of the user would be located to further aid in finger flexibility. FIG. 8 illustrates various embodiments in which the coil 705 is positioned in the channel 135 along the length of the finger member 120 from the base end 125 to the location of the orifices 150. FIGS. 9-12 illustrate cross-sectional views of the medical glove 100 that may be taken at any position along a length of the medical glove 100. FIG. 9 illustrates various embodiments in which the entire medical glove 100, including the portion comprising the channel 135, comprises a single material 905. The material 905 may be any material known in the art now or in the future for manufacturing medical gloves 100, such as latex, nitrile rubber, vinyl, neoprene, and the like. The channel 135 may be molded into the medical glove 100 as part of the manufacturing process. FIG. 10 illustrates various embodiments in which the medical glove 100 comprises a first material 905, and a portion of the medical glove 100 comprising the channel 135 comprises a second material 1005. The second material 1005 may be a more resilient composition than that of the first material 905 to provide additional structural strength to the sidewalls of the channel 135 such that the channel better resists collapsing when subjected to vacuum. The resilient second material 1005 may obviate the need to a reinforcing coil 705. Nonlimiting examples of the second material 1005 include polyisoprene or other resilient rubber compounds, silicone or other silicone-containing compounds, a fiber reinforced compound, a thermosetting polymer, or the like.

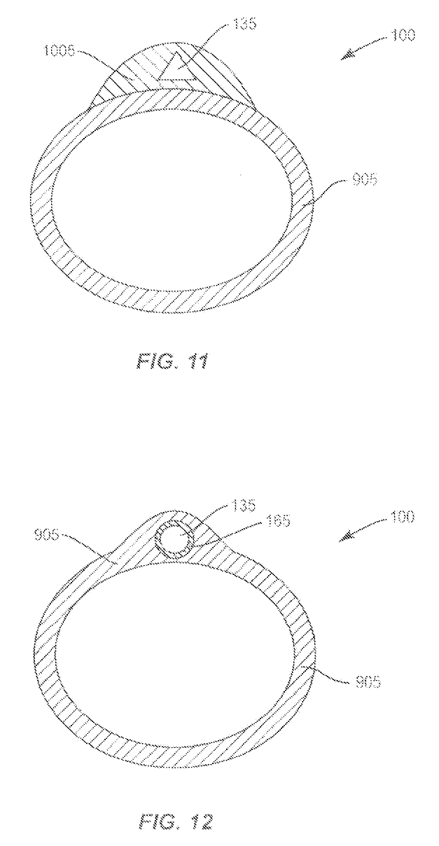

In the previous figures, the channel 135 is depicted as being generally round or oval. The scope of the present disclosure includes any shape channel 135 known in the art. For example, FIG. 11 illustrates various embodiments of a triangular shaped channel 135 formed in the second material 1005. The triangular shape, as well as other shapes not shown, may provide higher structural strength to resist sidewall collapse when the channel 135 is under suction. Different shapes of the channel 135 may also be advantageous for easier formation of the channel 135 during manufacture of the medical glove 100.

FIG. 12 illustrates suction tubing 165 imbedded with the glove material 905 (or the second material 1005) to form the channel 135. As illustrated previously in FIG. 1, the suction tubing 165 may extend beyond the cuff opening edge 160 and terminate at the connector 170 to facilitate connection to a suction source. The suction tubing 165 may extend from the cuff opening edge 160 up to (and including in certain embodiments) the orifices 150, or any portion of that length.

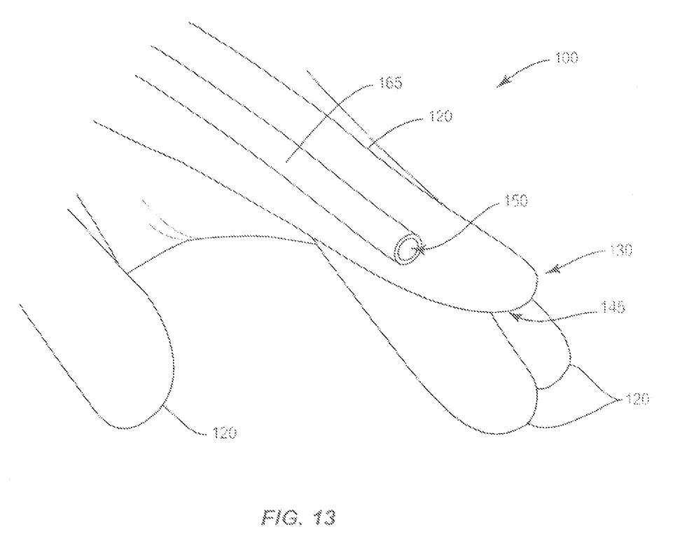

In certain situations, it may be advantageous to terminate the channel 165 before the tip end 130 of the finger member 120 as illustrated in FIG. 13 according to various embodiments. For example, the channel 165 may provide irrigation rather than suction, and placement of the source of the irrigation close to the tip end 130 may not be critical. In the example of FIG. 13, the channel 165 may terminate at a single orifice 150 designed to facilitate a flow of saline solution or other liquid.

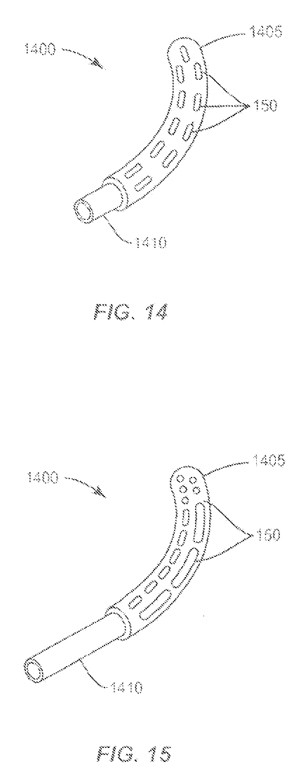

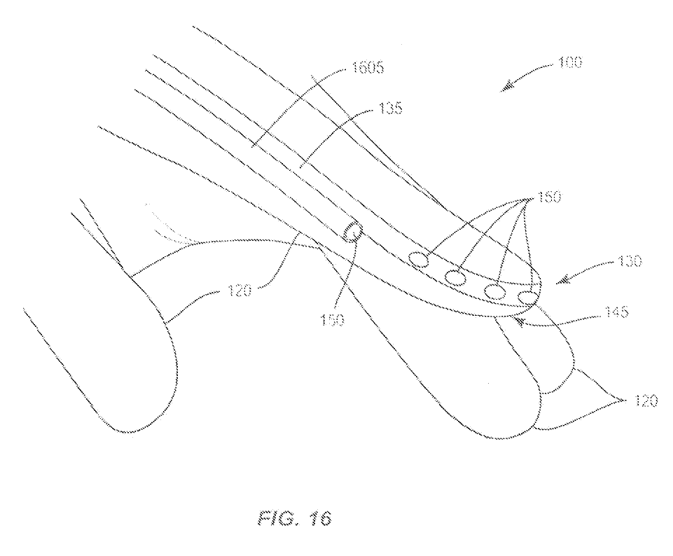

The embodiments illustrated in FIG. 13 may also facilitate the use of insertable suction tips 1400 as shown in FIGS. 14 and 15. The suction tips 1400 may comprise a suction head 1405 having one or more orifices 150, and a nipple 1410 adapted for releasable insertion into the channel 165 (such as the embodiment illustrated in FIG. 13). The nipple 1410 may be short as illustrated in FIG. 14 or longer as illustrated in FIG. 15 as is needed for specific applications. The insertable suction tips 1400 allow the user to select the proper orientation location, and size of orifices 150 for the specific application. For example, a dentist may initially choose the insertable suction tip 1400 shown in FIG. 14 with small orifices 150. During the procedure, the dentist may encounter more solid material to be suctioned away than originally anticipated, which may cause repeated clogging of the orifices 150. In this situation, the dentist may remove the first insertable suction tip 1400 and replace it with the insertable suction tip 1400 shown in FIG. 15 that has larger orifices 150 designed to handle increased amounts of solids. Additionally, the dentist may remove the first insertable suction tip 1400 to quickly clean out debris clogged in orifices 150 and may reattach the first insertable suction tip 1400 without needing to remove or replace the medical glove 100.

Although FIGS. 14 and 15 illustrate that the insertable suction tip 1400 is curved to conform to the tip end 130 of the finger member 120, any desired shape is within the scope of the present disclosure.

The previous figures have illustrated a single channel 135 running from the cuff opening edge 160 to a single finger member 120. FIG. 16 illustrates various embodiments comprising a second channel 1605. The second channel 1605 may, for example, provide an irrigation source while the first channel 135 provides suction.

Alternatively, the second channel 1605 may provide a second suction source. The second channel 1605, if used as a second suction source, may join with the first channel 135 at any point along the cuff 105, body section 110, or finger section 115. Alternatively, the second channel 1605, may be entirely separate from the first channel 135, terminating at the cuff opening edge 160. Any number of common or separate channels, 135, 1605 directed to any or all the finger members 120 on either a left-handed or right-handed medical glove 100 are understood to be within the scope of the present disclosure, regardless of how arranged on the medical glove 100.

In some embodiments, a primary orifice on the glove at the tip of the index finger provides the main suction, with one or more relief valve side holes in the integrated channel to facilitate suction without full force in delicate procedures. In other embodiments, the sides of the suction channel may be reinforced (or built up with what is referred to as a Triangular Suction Matrix) to prevent collapse under suction.

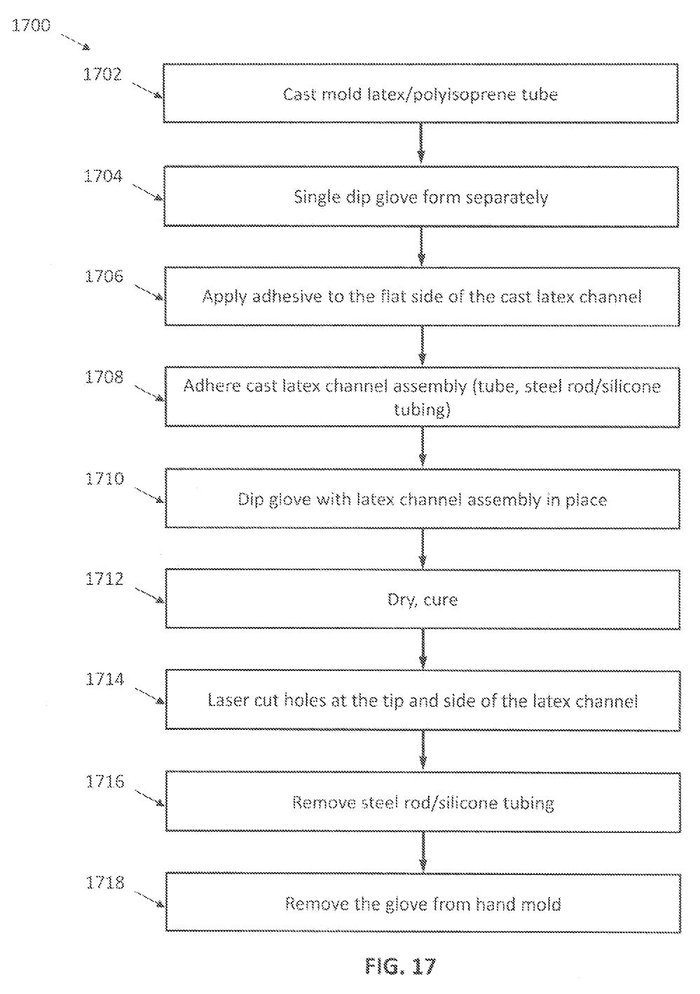

FIG. 17 is a flow diagram depicting a method 1700 for fabricating a medical glove with an integrated channel. As shown in FIG. 17, the method initially at 1702 cast-molds a latex or polyisoprene, or any other suitable synthetic polymer tube. Next, at 1704 the method separately single-dips the glove form. At 1706, adhesive is applied to the flat side of the cast latex channel. In some embodiments, an adhesive may not be required at 1706. At 1708, the cast latex channel assembly (i.e. a tube, steel rod/silicone tubing) is adhered to the glove. Next, at 1710, the method dips the glove with the latex channel assembly in place. Drying and curing is accomplished at 1712, while at 1714 holes are laser cut at the tip and in the side of the latex channel. Next, at 1716 the steel rod/silicone tubing is removed (rod may be removed before or after hand former dip). Finally, at 1718, the glove is removed from the hand mold.

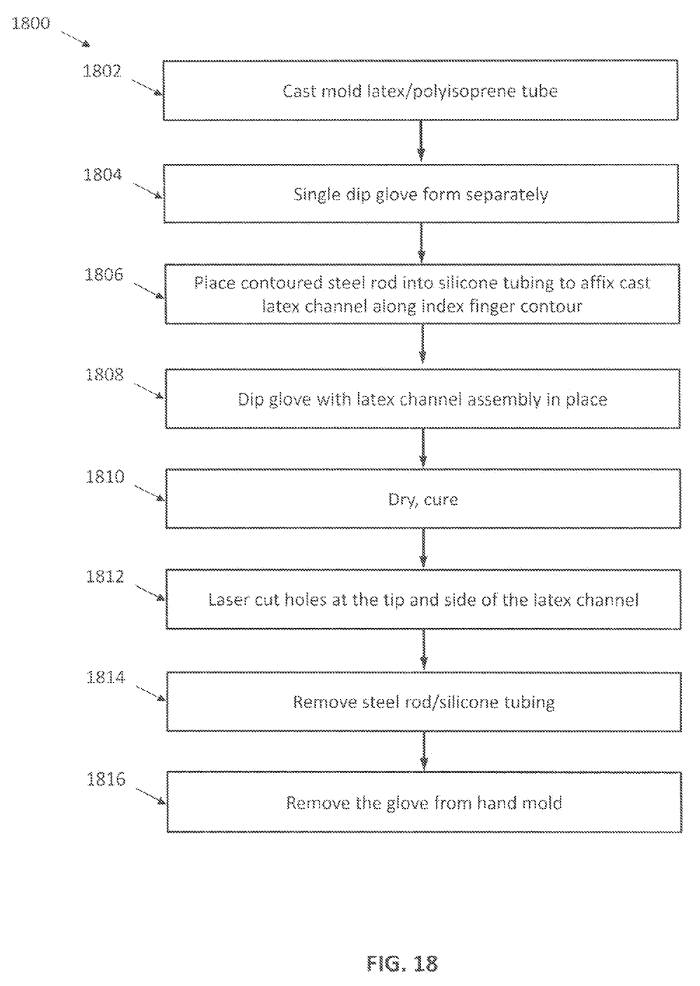

FIG. 18 is a flow diagram depicting an alternate method 1800 for fabricating a medical glove with an integrated channel. As shown in FIG. 18, the method initially at 1802 cast-molds a latex, polyisoprene or any other suitable synthetic polymer tube. Next, at 1804 the method separately single-dips the glove form. At 1806, a contoured steel rod is placed into silicon tubing to affix the cast latex channel along the index finger contour of the glove. It should be noted that in some embodiments the steel rod may be placed into the silicone before the latex tubing is cast at 1806. Next, at 1808, the method dips the glove with the latex channel assembly in place. Drying and curing is accomplished at 1810, while at 1812 holes are laser cut at the tip and in the side of the latex channel. Next, at 1814 the steel rod/silicone tubing is removed. Finally, at 1816, the glove is removed from the hand mold.

In some embodiments, the channel 135 may include one or more biosensors that may be used to detect and/or analyze biomarkers. In one embodiment, the biosensors may be in wireless communication with one or more computing devices using any wireless protocol, including IEEE 802.11 and Bluetooth. In other embodiments, the biosensors may be hard wired to one or more computing devices. In some embodiments, the biosensors may be configured to measure chemistries, blood and blood loss and concentrations, cellular content, distinguish between compositions of proteins, and/or identify pathogens.

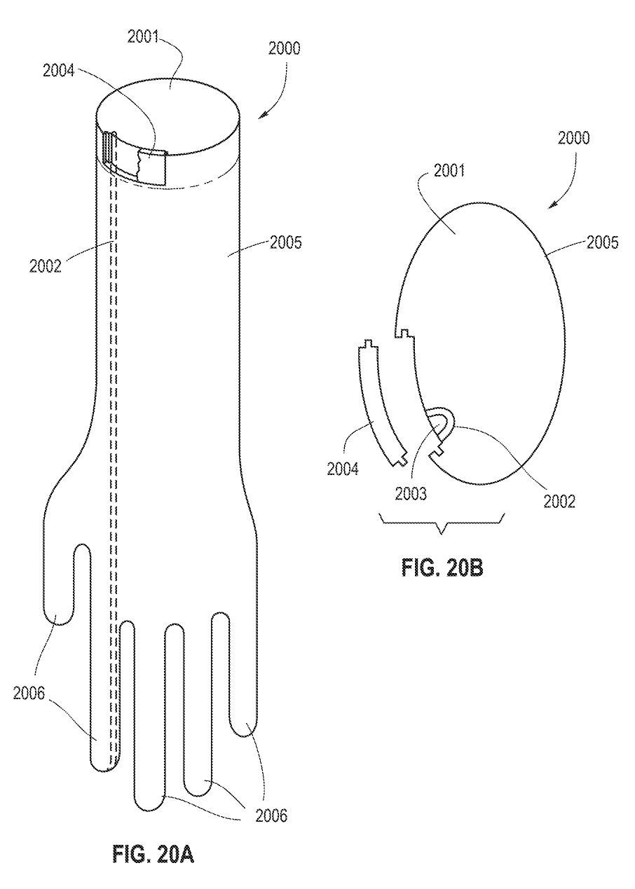

FIGS. 20A and 20B show a hand former 2000 for forming a medical glove, such as glove 100, with a pre-formed groove 2002 in an example. The hand former 2000 may include a body 2005 in substantially the shape of a hand with an opening 2001. A channel 2003 may be formed in the groove 2002. The glove may be formed form other materials, such as any synthetic polymers. A sleeve or cover plate 2004 may optionally be placed around all or part of the opening 2001 of the hand former 2000 in some embodiments. The groove 2002 may extend from the opening 2001 substantially to or near the end of one or more fingers 2006 of the hand former

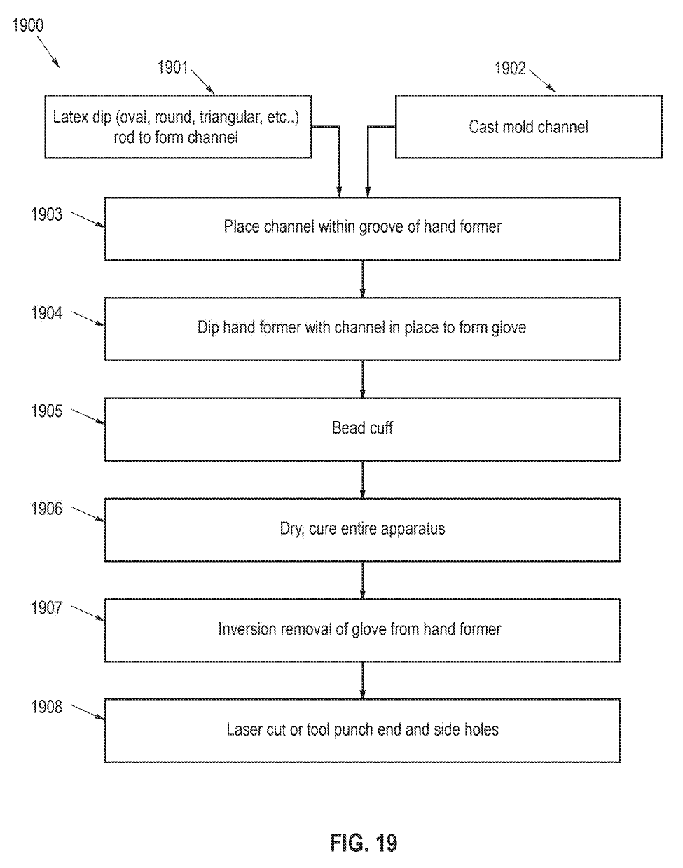

FIG. 19 shows a method 1900 for fabricating a glove using the hand former 2000 in an embodiment. The method 1900 may include at 1901 a latex dip of rod 2003 to form channel 2002. In one embodiment, the rod 2003 may extrude when forming the channel using injection molding. The rod 2003 may be any suitable shape including a circle, half circle, oval, triangle or square. Alternatively, at 1902, the mold channel may be cast. At 1903, the channel may be placed within a groove of the hand former. At 1904, a glove is formed by dipping the hand former with the channel in place. At 1905, the cuff of the hand former may be beaded. In one embodiment, the beading may be done while the hand former is horizontal and/or spinning. At 1906, the hand former may be dried and cured. At 1907, inversion removal of the glove from the hand former may be performed. Alternatively, at 1907, the glove may be removed using an air blowing process. At 1908, laser cutting or tool punching may be performed, for example, to form open end 145 or orifices 150.

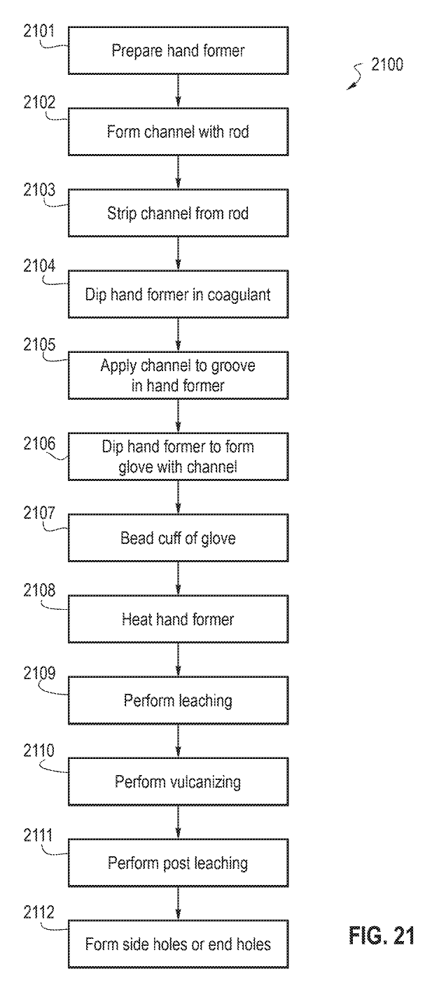

FIG. 21 shows another method 2100 for forming a glove, such as glove 100, using a hand former. The hand former may be substantially the same or different from hand former 2000. At 2101, a hand former may be cleaned and prepped, including water rinses, bleach rinses, and brushing. At 2102, a channel is formed with latex or synthetic material from dipping, extrusion, or cast mold on silicone, injection molding, ceramic or steel rod. The rod 2003 may be any suitable shape including a circle, half circle, oval, triangle or square. At step 2103, the channel may be stripped from the rod. At step 2104, the hand former may be dipped in coagulant. In one embodiment, the coagulant may have a temperature-controlled bath of approximately 70-85° C. At step 2105, the channel may then be applied within a groove of the hand former. At step 2106, the hand former may then undergo a latex or synthetic material dip to form the glove portion integrating with the channel. At step 2107, the cuff of the glove may be beaded. In one embodiment, the beading may be done while the hand former is horizontal and/or spinning. At step 2108, the hand former may be heated by air drying, while hand is horizontal and/or spinning. At step 2109, leaching may occur. At step 2110, vulcanizing may occur. At step 2110, post-leaching may occur. At step 2111, side holes or end holes at the distal end, such as orifices 150, may be laser cut or punched. At step 2112, inversion stripping of the glove from the hand former may occur. It should be noted that steps 2108-2112 are each optional and some or all of steps 2108-2112 may be performed.

The foregoing description of the preferred embodiments of the present invention has been presented for purposes of illustration and description. It is not intended to be exhaustive or to limit the invention to the precise form or to exemplary embodiments disclosed. Obviously, many modifications and variations will be apparent to practitioners skilled in this art. Similarly, any process steps described might be interchangeable with other steps in order to achieve the same result. The embodiment was chosen and described in order to best explain the principles of the invention and its best mode practical application, thereby to enable others skilled in the art to understand the invention for various embodiments and with various modifications as are suited to the particular use or implementation contemplated. It is intended that the scope of the invention be defined by the claims appended hereto and their equivalents. Reference to an element in the singular is not intended to mean “one and only one” unless explicitly so stated, but rather means “one or more.” Moreover, no element, component, nor method step in the present disclosure is intended to be dedicated to the public regardless of whether the element, component, or method step is explicitly recited in the following claims. No claim element herein is to be construed under the provisions of 35 U.S.C. Sec. 112, sixth paragraph, unless the element is expressly recited using the phrase “means for . . . . ”

Furthermore, the purpose of the foregoing Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The Abstract is not intended to be limiting as to the scope of the present invention in any way. It is also to be understood that the steps and processes recited in the claims need not be performed in the order presented.

1. A medical glove, comprising: a cuff having an opening for hand entry and an opening edge;
a body section coupled to the cuff; a plurality of finger members coupled to the body section generally opposite from the cuff, a first channel integrally formed in the glove, the first channel comprising: a first open end proximate to the opening edge; a second open end proximate to the tip end of one of the finger members; and an enclosed hollow space connecting the first open end and the second open end wherein the second open end comprises a plurality of orifices and at least one biosensor included in the channel.

2. The medical glove of claim 1, wherein the second open end extends at least partially around the tip end of the finger member.

3. The medical glove of claim 1, wherein the glove further comprises a first material that forms the channel.

4. The medical glove of claim 1, wherein one of each of the plurality of orifices has generally the same shape and at least one of the plurality of orifices has a different shape or size than the other orifices.

5. The medical glove of claim 1, wherein at least a portion of the orifices are arranged in two or more rows.

6. The medical glove of claim 1, further comprising a second channel.

7. The medical glove of claim 6, wherein the second channel is positioned on one of the same finger member as the first channel and a different finger member than the first channel.

8. The medical glove of claim 6, wherein the first channel provides suction and the second channel provides irrigation.

9. The medical glove of claim 1, further comprising one or more supporting structures to prevent collapse of the first channel when the first channel is under suction

10. The medical glove of claim 1, wherein the first channel further comprises a tube embedded into the glove.

11. A method of forming a medical glove, comprising:
forming a channel;
placing the channel into a groove of a hand former, the groove configured for receiving the channel;
beading a cuff of the hand former;
drying or curing the hand former;
removing the formed glove from the hand former;
laser cutting or machine punching one of end and side holes in the glove.

12. The method of claim 11 further comprising one of leaching and vulcanizing the hand former.

13. The method of claim 11 further comprising forming the glove from one of latex and a synthetic polymer.

14. The method of claim 11 further comprising forming the channel from one of dipping, extrusion, cast mold on silicone, injection molding, ceramic and a steel rod.

15. The method of claim 11 further comprising including a biosensor in the channel.

16. The method of claim 11 further comprising one of analyzing and detecting biomarkers with the biosensor

17. A hand former for a medical glove, comprising:
a body substantially in the shape of a hand;
an opening at one of end of the body; and
a groove extending from the opening substantially to or near the end of one or more fingers of the hand former and configured to receive a channel.

18. The hand former of claim 11, wherein the medical glove is formed from one of latex and a synthetic polymer.

19. The hand former of claim 11, wherein the channel is formed from one of dipping, extrusion, cast mold on silicone, injection molding, ceramic and a steel rod.

20. The hand former of claim 11, wherein a biosensor is included in the channel.
